(19) United States
(12) Patent Application Publication
(10) Pub. No.: US 2013/0035258 A1
(43) Pub. Date: Feb. 7, 2013

(54) LUMINESCENT TETRAPODS DOTS AND VARIOUS APPLICATIONS

(71) Applicants: Krishnan Chakravarthy, Williamsville, NY (US); Siddhartha Venkata Kamisetti, Cleveland, OH (US)

(72) Inventors: Krishnan Chakravarthy, Williamsville, NY (US); Siddhartha Venkata Kamisetti, Cleveland, OH (US)

(73) Assignee: NANOAXIS, LLC, Clarence, NY (US)

(21) Appl. No.: 13/567,078

(22) Filed: Aug. 5, 2012

Related U.S. Application Data
(60) Provisional application No. 61/515,468, filed on Aug. 5, 2011.

(51) Int. Cl. G01N 21/64; C12Q 1/70; C40B 40/00; C12Q 1/68; G01N 33/53; C12Q 1/02

(52) U.S. Cl. 506/13; 435/7.2; 435/29; 435/6.12; 435/7.1; 436/501; 435/5

(57) ABSTRACT

A tetrapod article and its uses are disclosed whereby such tetrapod article comprises an apex base nanomaterial and X leg base nanomaterial protruding from the apex, wherein X is an integer. In an aspect, the tetrapod article can comprise Y nanomaterial layers overcoating the apex base material and the leg base nanomaterial protruding from the apex, wherein Y is an integer. In yet another aspect, disclosed is an assay kit comprising a set of hydrophobic coated tetrapod articles coupled to K pairing moieties that are paired to L predefined targeting items to form a set of tetrapod conjugates that that correspond to M biological parameters and luminesce at respective wavelengths that correspond to the respective predefined target cell types, wherein K, L, and M are integers.

### CROSS REFERENCED TO RELATED APPLICATION

This application claims the benefit of U.S. Provisional Application No. 61/515,468, filed Aug. 5, 2011, and entitled “Luminescent Tetrapod Dots and Various Applications”, the entirety of which is incorporated herein by reference.

### TECHNICAL FIELD

This disclosure relates to a compound with the structure of a tetrapod (herein referred to as a “tetrapod dot” or “tetrapod dots” in the plural) which can be made of one or more nanomaterials. A tetrapod dot is capable of binding to one or more particles, materials, genetic materials or materials with biological properties and may be used for diagnostic tetrapod-based immunoassays for detecting biological substances such as proteins, DNA, RNA, PNA (polynucleotide nucleic acid), microRNA, antigen, antibody, receptor, ligand, sugar, and other such biological substances. A tetrapod dot has numerous characteristics such as the ability to luminescence and can be used in multiplexing applications to identify numerous targets.

### BACKGROUND

In order to advance the field of medicine and research, a great deal of importance is placed on the ability to diagnose and detect biological substances (such as an antigen or cellular receptor associated with a malignant cell). In many instances, an assay kit can be utilized to detect biological substances (e.g. cellular receptors) and for various diagnostic purposes, such as, to count cells, measure cells, measure cell constituents, measure cell granularity, utilize fluorescence as a detection modality and conduct other research and development activities. In some instances, assay kits are used to detect and diagnose through use of fluorescent labeling in order to assist a user in identifying a specific type of cell. All of these purposes are of great interest to researchers, developers, medical practitioners, and other assay kit users.

Traditional assays utilize fluorescent labels such as organic dyes, rhodamine, or fluorescein. The detection of cells with fluorescent labeling techniques such as organic dye molecules, make use of the molecule by attaching it to a biological substance, whereby the organic dye molecule luminesces when the dye is excited. Unfortunately, such fluorescent labels can only detect a limited number of biological parameters associated with the biological substance due to poor factors including wide tail of emission spectrums, broad wavelengths, poor photostability and a limitation of colors leading to the detection of a few biological parameters at a single time. Better fluorescent labels are needed which can detect numerous biological parameters in a biological substance simultaneously.

### SUMMARY

The following presents a simplified summary of the disclosure in order to provide a basic understanding of some aspects of the disclosure. This summary is not an extensive overview of the disclosure. It is intended to neither identify key or critical elements of the disclosure nor delineate any scope particular embodiments of the disclosure, or any scope of the claims. Its sole purpose is to present some concepts of the disclosure in a simplified form as a prelude to the more detailed description that is presented later.

In accordance with one or more embodiments and corresponding disclosure, various non-limiting aspects are described in connection with tetrapod articles, tetrapod-based assay kits for the detection of biological parameters, and other tetrapod based applications. In accordance with a non-limiting embodiment, in an aspect, a tetrapod article, comprising: an apex base nanomaterial; and X leg base nanomaterial protruding from the apex, wherein X is an integer is disclosed. In an aspect, the tetrapod article comprises Y nanomaterial layers overcoating the apex base material and the leg base nanomaterial protruding from the apex, wherein Y is an integer.

In another aspect, the tetrapod article is comprised of an apex with four leg base nanomaterials protruding from the apex. In an instance, the apex or the leg is coated with a hydrophilic coating. Furthermore, in an aspect, the apex base nanomaterial is at least one of CdS, CdSe, MgSe, MgTe, MgS, ZnS, ZnTe, ZnSe, CaS, CaSe, SrS, SrSe, SrTe, HgS, HgSe, HgTe, BaS, BaSe, BaTe, or CdTe. In an instance the nanomaterial layers is at least one of CdS, CdSe, MgSe, MgTe, MgS, ZnS, ZnTe, ZnSe, CaS, CaSe, SrS, SrSe, SrTe, HgS, HgSe, HgTe, BaS, BaSe, BaTe or CdTe. In another aspect, an assay kit comprising: a set of hydrophilic coated tetrapod articles coupled to K pairing moieties that are paired to L predefined targeting items to form a set of tetrapod conjugates that that correspond to M predefined biological parameters and luminesce at respective wavelengths that correspond to the respective predefined target cell types, wherein K, L, and M are integers is disclosed.

In yet another aspect, the tetrapod article comprises an apex base nanomaterial paired to both ends of each respective leg base nanomaterial. In an aspect, the pairing moiety is any one of a thiol moiety, F127COOH, alkyl group, propyl group, N-(3-aminopropyl)-3-mercapto-benzamide, 3-aminopropyl-trimethoxysilane, 3-mercaptopropyltrimethoxysilane, 3-maieimidopropyl-trimethoxysilane, or 3-hydrazidopropyl-trimethoxysilane, diacetylenes, acrylates, acrylamides, vinyl, and styryl. In an aspect, the pairing moiety is any one of a thiol moiety, F127COOH, alkyl group, propyl group, N-(3-aminopropyl)-3-mercapto-benzamide, 3-aminopropyl-trimethoxysilane, 3-mercaptopropyltrimethoxysilane, 3-maleimidopropyl-trimethoxysilane, or 3-hydrazidopropyl-trimethoxysilane, diacetylenes, acrylates, acrylamides, vinyl, and styryl.

In another aspect, the targeting item is any one of an antibody, a nucleic acid, a protein, a polysaccharide, a small molecule, avidin, streptavidin, a biotin, a antidigoxiginen, a monoclonal antibody, a polyclonal antibody, a nucleic acid, monomeric nucleic acid, 40 oligomeric nucleic acid, a protein, a polysaccharide, a sugar, a peptide, a drug, carbohydrate, ligands. In an aspect, the targeting item is any one of an antibody, a nucleic acid, a protein, a polysaccharide, a small molecule, avidin, streptavidin, a biotin, a antidigoxiginen, a monoclonal antibody, a polyclonal antibody, a nucleic acid, monomeric nucleic acid, 40 oligomeric nucleic acid, a protein, a polysaccharide, a sugar, a peptide, a drug, carbohydrate, ligands. In yet another aspect, the tetrapod article is synthesized by a flow chemistry micro-reactor process.

### BRIEF DESCRIPTION OF THE FIGURES

FIG. 1 illustrates an example non-limiting tetrapod article

FIG. 2 illustrates an example non-limiting tetrapod article and its adjustable properties.

FIG. 3 illustrates an example non-limiting tetrapod article and respective spectral signatures.

FIG. 4 illustrates an example non-limiting tetrapod article and a nanomaterial layering overcoat.

FIG. 5 illustrates an example non-limiting tetrapod article and a nanomaterial layering overcoat.

FIG. 6 illustrates an example non-limiting tetrapod article and a pairing moiety.

FIG. 7 illustrates an example non-limiting tetrapod article and its associated pairing.

FIG. 8 illustrates an example non-limiting tetrapod article based assay kit.

FIG. 9 illustrates an example non-limiting tetrapod article based assay kit and multiplexing.

FIG. 10 illustrates an example non-limiting tetrapod article and a pairing moiety whereby the pairing moiety is paired to a targeting item.

FIG. 11 illustrates an example non-limiting tetrapod article and a pairing moiety whereby the pairing moiety is paired to a targeting item that is bound to a biological parameter and predefined target.

FIG. 12 illustrates an example non-limiting tetrapod dots and mechanisms for synthesizing tetrapod dots that emit at varying wavelengths.

FIG. 13 illustrates an example non-limiting tetrapod article and its use in a sandwich ELISA.

FIG. 14 illustrates an example non-limiting tetrapod article scale-up production technique.

FIG. 15 illustrates an example non-limiting graph of tetrapod articles absorbance wavelength, emission wavelength and excitation wavelength of the excitation source.

FIG. 16 illustrates an example non-limiting graph of tetrapod articles bioconjugated to folic acid by plotting the absorbance on the y-axis and the wavelength on the x-axis.

FIG. 17 illustrates an example non-limiting graph of the fluorescence of the tetrapod articles bioconjugated to folic acid.

FIG. 18 illustrates an example non-limiting graph of the fluorescence of tetrapod articles bioconjugated to folic acid over a period of time.

### DETAILED DESCRIPTION

### Overview

A tetrapod article is a unique nanoscale compound which is a composite of one or more nanomaterials and can take the shape of a tetrapod. The tetrapod article is capable for use in various biological applications due to its material composition and its compatibility with biological materials. Further, the luminescent compound may be used for many diagnostic and imaging purposes and specifically possesses properties, which lend it to multiplexing. Through various nanomaterial compositions, various respective arm lengths, various respective arm widths, various respective apex diameters, various respective apex sizes, each tetrapod article is able to possess attributes wherein respective components of said tetrapod article may emit energy at various wavelengths thereby serving to identify over one hundred parameters for various target applications.

Tetrapod articles are small, nanoparticles, which comprise sizes in the 0.001 to 999.99 nm range. These nanoparticles are attractive for a variety of uses in a variety of applications due to its adjustability and its inherent luminescent properties. Tetrapod articles may be synthesized in a variety of sizes, shapes, and material compositions (e.g. tetrapod articles may be comprised of metal, polymer, semiconductor). Tetrapod dots can be synthesized in a variety of ways, such as branched growth of one or more arms (e.g. four arms branching in the case of a tetrapod dot) from a central axis.

In an aspect, the tetrapod dot is capable for use in various biological applications due to its material composition and compatibility with biological materials. Further, the luminescent compound may be used for many diagnostic and imaging purposes and specifically contains properties, which allow for its use in multiplexing applications. Through the synthesis of tetrapod dots of various nanomaterial compositions, various arm lengths, various arm widths, various apex diameters, and various apex sizes, each tetrapod is able to possess attributes wherein unique tetrapod dot can emit energy at a unique wavelength thereby serving to identify numerous parameters for various diagnostic applications.

One use for luminescent tetrapod dots are for production of assay kits comprised of surface modified luminescent tetrapod dots that correspond to desired biological targets of a desired concentration which luminesce when exposed to an excitation source. Accordingly, each biological target can be tagged by a unique tetrapod dot of unique characteristics (e.g. unique spectral signature, unique emission wavelength, and so on). Another application for tetrapod dots is its use as a therapeutic delivery vehicle wherein the tetrapod dots may be chemically modified to carry and deliver a drug or biological material to a specific destination within a subject (e.g. an animal or human) for therapeutic treatment purposes. In such an instance, the tetrapod dots are an ideal ‘theranostic’ material that can both be imaged and can deliver a therapeutic payload to a target.

### Example Embodiments of Tetrapod Dots, Assay Kits and Tetrapod Dot Applications

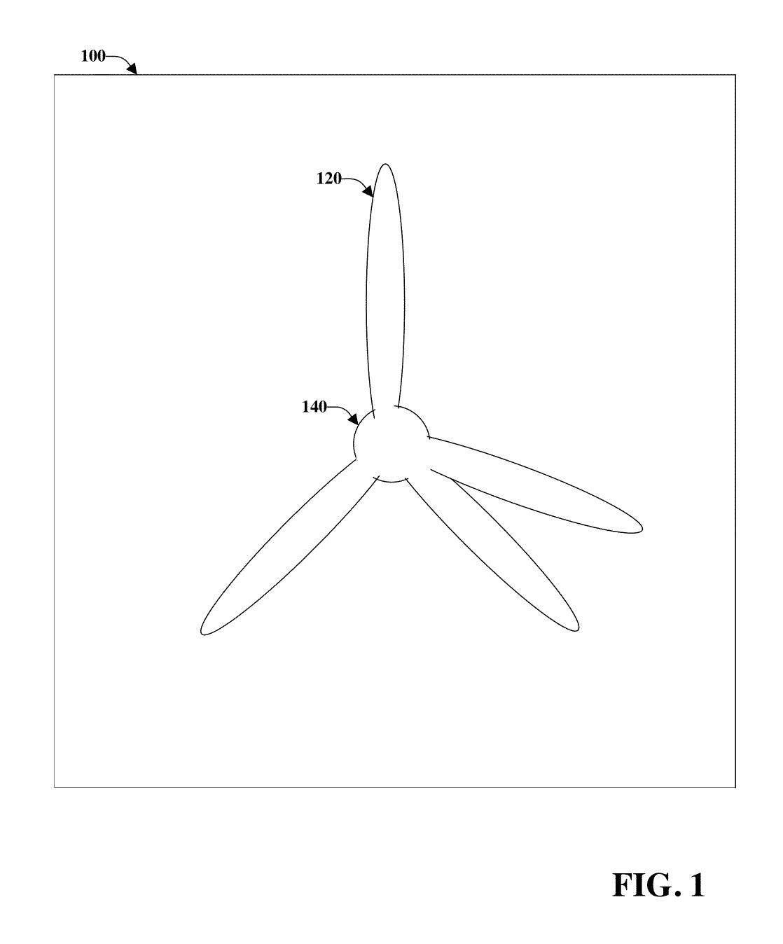

Referring now to the drawings, with reference initially to FIG. 1, tetrapod article 100 is shown. Aspects of the tetrapod dots, products, assay kits, tetrapod dot applications, synthesis methods and other such disclosed references explained in this disclosure can constitute one or more embodiments with one or more components or features.

In an embodiment, tetrapod article 100 comprises an apex base nanomaterial; and X leg base nanomaterial protruding from the apex, wherein X is an integer. In an aspect, tetrapod article 100 is comprised of apex 140 and leg 120 base nanomaterials protruding from the apex 100. In an instance, X is equal to four in the case of tetrapod article 100. Accordingly, four leg 140 protrude from apex 100 for purposes of tetrapod article 100. However, a tetrapod article can possess one leg 140 (e.g. X=1) protruding from apex 140 or possess thirty five leg 140 (e.g. X=35) because X can be any integer.

In an aspect, tetrapod article 100 can comprise an apex 120 base nanomaterial and a leg 140 base nanomaterial. A base nanomaterial can be a structured semiconductor nanomaterial with the ability to absorb and emit a energy such as UV light (e.g. electromagnetic radiation, UV light, etc.). In an aspect, once energy is absorbed by a base nanomaterial, the base nanomaterial can scatter the energy, light or electromagnetic radiation subsequent to its excitement by an excitation source such as an electromagnetic radiation source, UV light source, or a particle beam which can demonstrate a detectable and measurable change in absorption and emission of radiation, UV light or energy in a narrow wavelength band. In an aspect, an advantage to using tetrapod article 100 is that only one common source for excitation (e.g. UV light) can excite several tetrapod dots due to the broad bandwidth of the nanomaterials. Another way of describing this property is, one excitation source can cause several tetrapod dots to give off radiation at different wavelengths, thereby permitting simultaneous excitation.

The tetrapod article 100 is special in that when it is prepared with a specific shell preparation and surface chemistry, it can be used in many applications, particularly for biological diagnostic and therapeutic purposes as explained herein. The optical properties, material composition, shape and structure of the tetrapod article, allow for variance and flexibility in the properties of the tetrapod article. In an aspect, tetrapod article 100 is a type of luminescent quantum dot. Quantum dots can be of various forms, structures, shapes, material compositions and sizes. Although, we describe tetrapod article 100 in great detail, any shape and size of quantum dot may be used for many of the applications described herein, the tetrapod article 100 has unique characteristics and properties which make it more versatile and desirable for the applications described herein. In an instance, quantum dots can take the form of a snowflake with a high degree of branching, polytypic crystal structures (e.g. two or more crystal structures in different domains of the same crystal, branched inorganic nanostructures, arrow-shaped nanocrystal particles, tree shaped nanocrystal particles, branched tetrapods, monopods, bipods, tripods, rods, teardrops, disks, cubes, stars, pyramids, heterostructures, non-spherical-shaped nanocrystals.

In an aspect, the base nanomaterial of tetrapod article 100 can comprise a semiconductor material. The tetrapod article apex 120 base nanomaterial and leg 140 base nanomaterial can be a II-VI semiconductor or a III-V semiconductor. For instance, in an aspect, the apex 120 base nanomaterial can be comprised of any one of: cadmium, sulfur, selenium, or tellurium. Furthermore, in an aspect, the leg 140 base nanomaterial can be comprised of any one of cadmium, sulfur, selenium, or tellurium. In another aspect, the tetrapod article 100 can be of a size ranging from 0.001 nm to 999.99 nm. The size of the tetrapod article can be measured in a variety of ways such as by leg width, leg length, measurement from the end point of one leg to the end point of another leg, or the measurement from the apex 140 to the end point of leg 120. For instance, where X is four, a leg 120 of the tetrapod article 100 can have one leg of length 15 nm and width 4 nm, a second leg of length 10 nm and width 5 nm, a third leg of length 25 nm and width 7.5 nm, and a fourth leg of length 5 nm and width 3 nm. In an aspect, the leg length, width, and apex size can be adjusted to create unique tetrapod article 100. A tunneling electron microscope can be used to confirm the leg length and width.

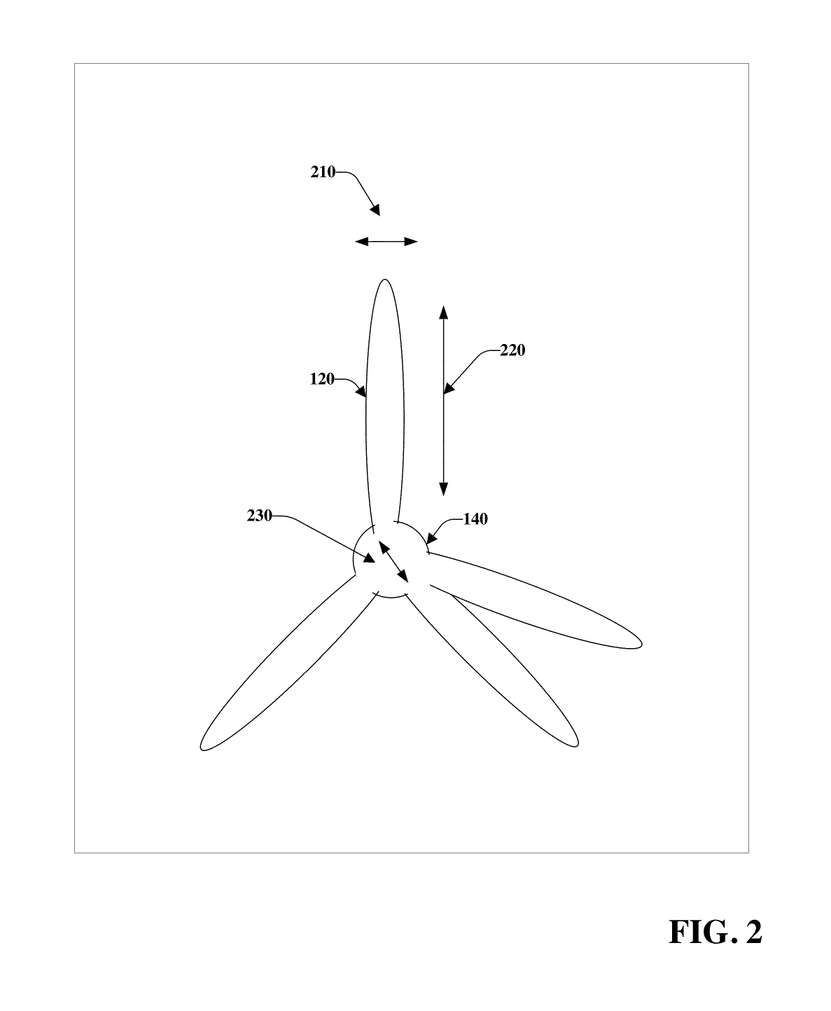

Turning now to FIG. 2, shown is tetrapod article 200, which demonstrates the adjustable features of a tetrapod article. In an aspect, tetrapod article 200 demonstrates the ability to adjust the leg 120 lengths longer or shorter as shown by double-sided arrow 220. In another aspect, tetrapod article 200 demonstrates the ability to adjust the leg 120 width longer or shorter as shown by double-sided arrow 210. Furthermore, in an aspect, tetrapod article 200 demonstrates the ability to adjust the apex size as larger or smaller as demonstrated by double-sided arrow 230. Each adjustable parameter 210, 220, and 230 allows for tetrapod article 200 to take on unique characteristics such as distinct spectral signatures.

A spectral signature is a read-out that describes the characteristic of each unique tetrapod. For instance, the tetrapod article 200 can be excited by a UV light source whereby tetrapod article 200 absorbs the UV light and subsequently emits the UV light at a wavelength. A spectral signature is a graphical representation of the UV light absorbed or emitted (or both absorbed and emitted as represented by two line graphs) by the tetrapod article 200. The spectral signature is indicated by a curve whereby the peak of the curve associates with a unique wavelength of each object identified. Therefore, each tetrapod article comprised of a unique combination or permutation of leg 120 width, leg 120 length, and apex size will display a unique spectral signature.

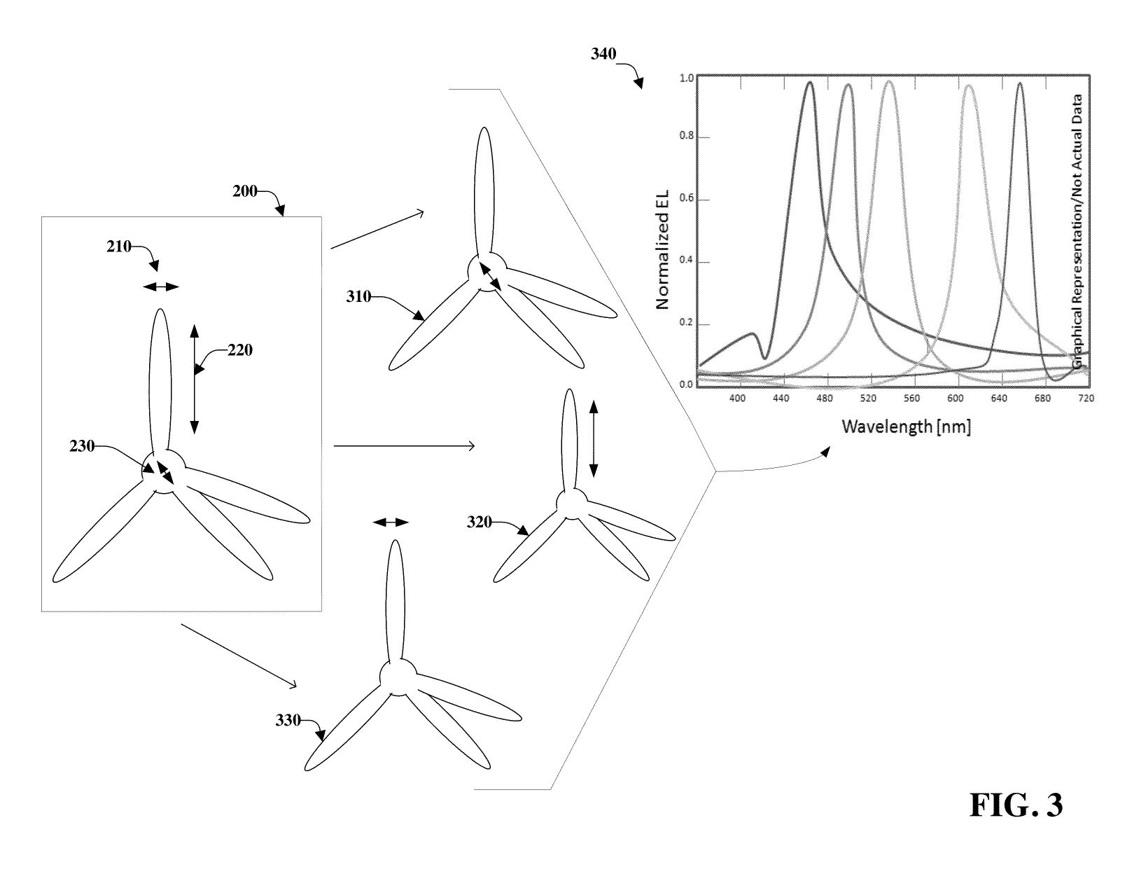

Turning now to FIG. 3, illustrated is the spectral signature of three different tetrapod articles after adjustment of the tetrapod articles. In an aspect, tetrapod article 200 can be adjusted to possess a smaller (or longer) apex as demonstrated at 230, which can result in a tetrapod article 310. Furthermore, tetrapod article 200 can be adjusted to possess a shorter (or longer) leg length as demonstrated at 220, which can result in a tetrapod article 320. In another aspect, tetrapod article 200 can be adjusted to possess a shorter (or longer) leg width as demonstrated at 210, which can result in a tetrapod article 330. These adjustments take place by synthesizing a new batch of tetrapod articles to possess unique characteristics (e.g. length, width, apex size) from the original tetrapod article.

The synthesis of unique tetrapod articles results in new tetrapod articles that possess unique spectral signatures, which can be demonstrated at graph 340. For instance by, adjusting the apex size tetrapod article 310 may emit at the wavelength peak shown by the red curve. In another aspect, by adjusting the leg length, the tetrapod article 320 may emit at a wavelength peak indicated by the green curve. Furthermore, in an aspect, by adjusting the leg width, the tetrapod article 330 may emit at a wavelength indicated by the dark blue curve. Each respective unique tetrapod can emit at a different wavelength peak and because the wavelengths possess a very narrow bandwidth as shown by graph 340, many distinct tetrapod articles may be identified and plotted on a single graph. These distinct spectral signatures allow for tetrapod articles to be used in a variety of applications including diagnostic assay kits and other such detection applications (e.g. ELISA, western blots, lateral flow assays, sandwich ELISA, peizoarray, microarray, flow cytometry applications, immunohistochemistry, microscopic applications, FRET, dual path platform, and other applications that use monoclonal and polyclonal antibodies for diagnostic applications).

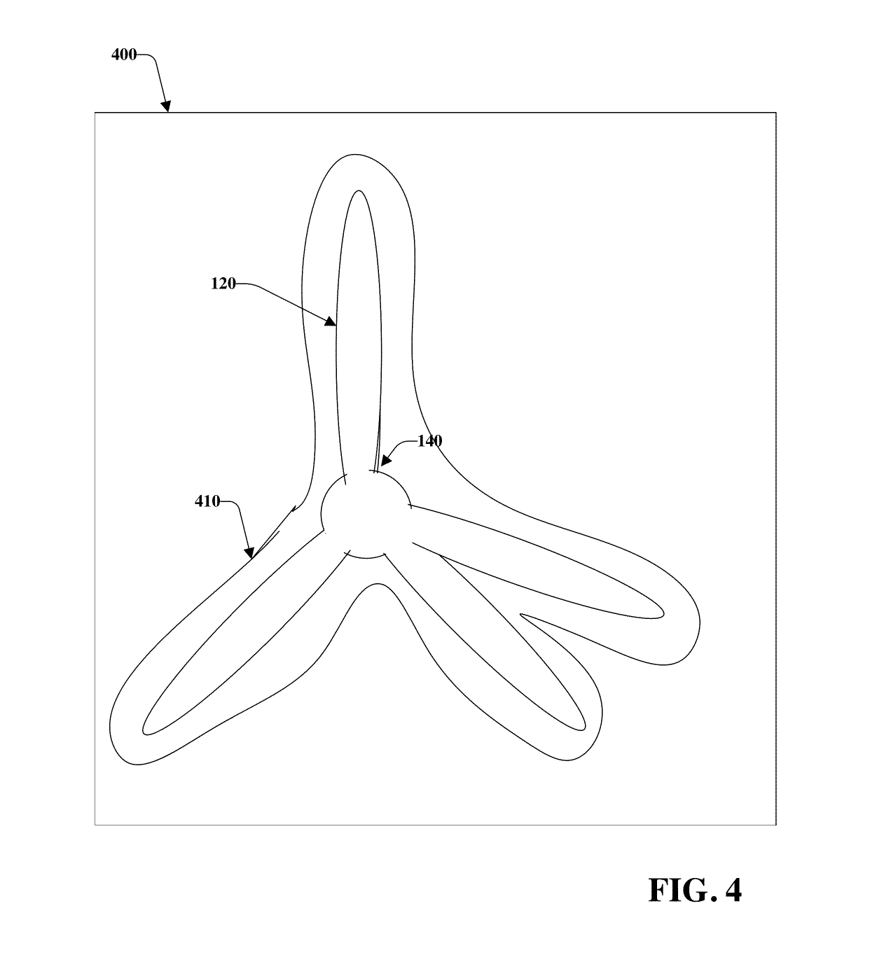

Turning now to FIG. 4, tetrapod article 400 is shown comprising Y nanomaterial layers 410 overcoating the apex 140 base material and the leg 120 base nanomaterial protruding from the apex 140, wherein Y is an integer. In an aspect, the apex 140 base nanomaterial and leg 120 base nanomaterial can be overcoated with one or more nanomaterial layers 410. A nanomaterial layer 410 can be an II-VI semiconductor or a III-V semiconductor. For instance, in an aspect the apex 140 base nanomaterial and the leg 120 base nanomaterial of a tetrapod article can be comprised of CdSe. Furthermore, a nanomaterial layer 410 that is CdS can overcoat the apex 140 base material and leg 120 base nanomaterial. Thus the tetrapod article can comprise a CdSe arm 140 and leg 120 base nanomaterial overcoated by a CdS nanomaterial layer. The layers can vary in thickness, and in some instance the overcoat can be a layer that is 1-30 monolayers in thickness.

The nanomaterial layer 410 can benefit the tetrapod article by furthering its shelf life, stability, providing for greater emission intensity in some instances, lessening blinking sometimes associated with the luminescence of tetrapod article, enhancing luminescence, enhancing photostability, and other such benefits. The nanomaterial layer 410 can also present less cellular toxicity depending on the composition of the apex 140 base material and leg 120 base material. By adding additional nanomaterial layer 410 the tetrapod article is less likely to degrade, whereby degradation could cause the base material to enter the surrounding environment of the tetrapod article (e.g. cellular matrix, blood stream). Thus the nanomaterial layer 410 can prevent intracellular toxicity thereby improving the utility of tetrapod articles for therapeutic uses in subjects. Furthermore, the more nanomaterial layer 410 surrounding the tetrapod article, the less likely cellular toxicity issues will arise.

In another aspect, the apex 140 base nanomaterial, leg 120 base nanomaterial or nanomaterial layer 410 can comprise a combination that is any one of a: Group 12, 13, 14, or 15 metal, Group II-VI semiconductor, Group II-V semiconductor, MgS, MgSe, MgTe, CaS, CaSe, CaTe, SrS, SrSe, SrTe, BaS, BaSe, BaTe, ZnS, ZnSe, ZnTe, CdS, CdSe, CdTe, HgS, HgSe, HgTe, GaAs, InGaAs, InP, InAs or metalloid. For instance, the apex 140 base nanomaterial and leg 120 base nanomaterial can comprise any one of a: Group 12, 13, 14, or 15 metal, Group II-VI semiconductor, Group II-V semiconductor, MgS, MgSe, MgTe, CaS, CaSe, CaTe, SrS, SrSe, SrTe, BaS, BaSe, BaTe, ZnS, ZnSe, ZnTe, CdS, CdSe, CdTe, HgS, HgSe, HgTe, GaAs, InGaAs, InP, InAs or metalloid. Furthermore, the nanomaterial layer 410 overcoating can comprise any one of a Group 12, 13, 14, or 15 metal, Group II-VI semiconductor, Group II-V semiconductor, MgS, MgSe, MgTe, CaS, CaSe, CaTe, SrS, SrSe, SrTe, BaS, BaSe, BaTe, ZnS, ZnSe, ZnTe, CdS, CdSe, CdTe, HgS, HgSe, HgTe, GaAs, InGaAs, InP, InAs or metalloid.

In yet another aspect, the nanomaterial layer 410 overcoating can comprise any one or more of a variety of biocompatible surface coatings such as ligand exchange, amphiphile encapsulation, and amphiphilic polymer coatings, as well as other coatings. Ligand exchange means overcoating the surface of the apex 140 base nanomaterial or leg 120 base nanomaterial or nanomaterial layer 140 overcoating with bifunctional capping molecules (e.g. 1-thioglycolic acid, 1-thioglycerol, mercaptoethylamine, L-cysteine, 3-mercaptopropionic acid, N-acetyle-L-cysteine, dihydrolipoic acid, . . . ). In another aspect, the surface of the apex 140 base nanomaterial or leg 120 base nanomaterial or nanomaterial layer 140 can be overcoated with an amphiphile encapsulation, which occurs when a tetrapod article is encapsulated in an amphiphile (e.g. DSPE phospholipids, 1,2-distearoyl-sn-glycero-3-phosphoethanolamine-N-[methoxy[poly(ethylene glycol)]], DSPEmPEG 5000, 1,2-distearoyl-sn-glycero-3-phosphoethanolamine-N-[amino(poly(ethylene glycol))2000], DSPE-PEG 2000 Amine, cyclodextrin). These coatings can transfer the tetrapod article into water (e.g. allow for soluble dispersion of the tetrapod article in aqueous solution). Thus, in an aspect, the apex 140 or the leg 120 is coated with a hydrophilic coating. In yet another aspect, the nanomaterial layers 140 overcoating is a hydrophilic coating. Various methods can be used to synthesize tetrapod articles in an aqueous medium.

In an aspect, the nanomaterial layers 410 can be any one or more of: Pluronic F127, silicon, micelle, glass, polymeric oxide, oxide of phosphorus, or polymeric ligands, amphiphilic ligand, or hydrophilic thiol compound. With respect to some biological applications, the tetrapod article requires pairing to an associated biological molecule. To address this function the tetrapod articles need to be converted from their original hydrophobic exterior to an hydrophilic exterior. This can be either accomplished using various overcoating methods or through direct one-pot synthesis within water. This will then allow for associated pairing moieties to pair to targeting items such as antibodies, nucleic acids, polysaccharides, proteins, drugs, monoclonal antibodies, polyclonal antibodies, and other such targeting items.

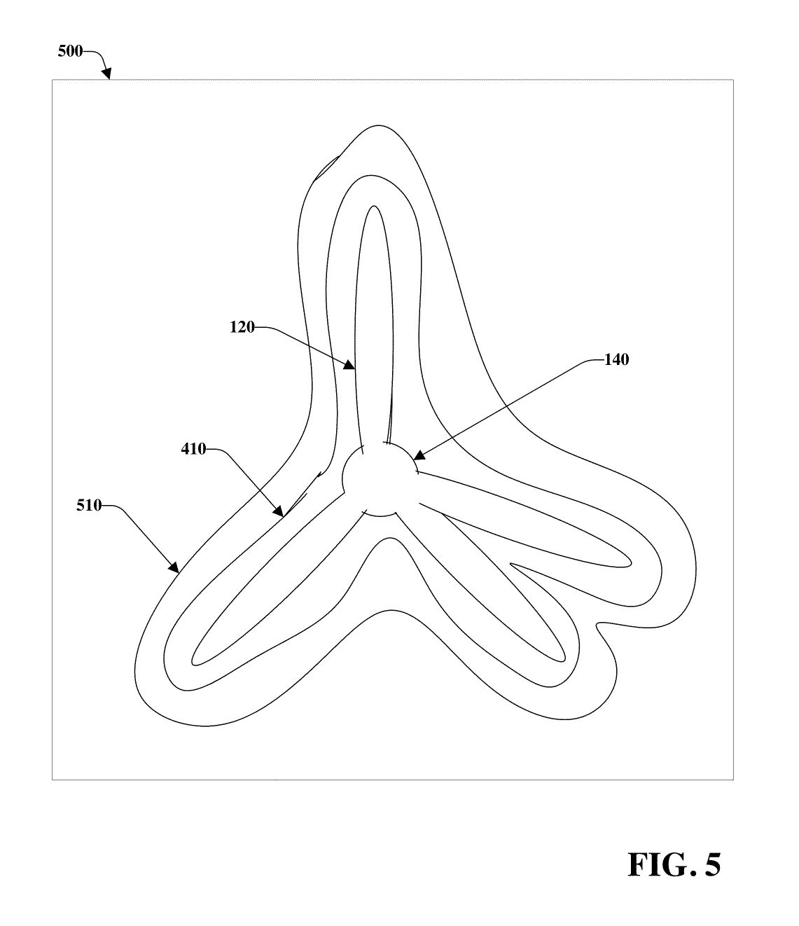

Turning now to FIG. 5, shown is tetrapod article 500 that demonstrates a second nanomaterial layer 510 that overcoats a nanomaterial layer 410 that overcoats the apex 140 base nanomaterial and leg 120 base nanomaterial. Tetrapod article 500 demonstrates how Y is an integer, whereby Y in this instance is two. Thus two overcoats encapsulate the apex 140 base nanomaterial and leg 120 nanomaterial in FIG. 5. Accordingly nanomaterial layer 510 can be a hydrophilic coating, a semiconductor material or any other such material described as nanomaterial 410 above. In an aspect, other such layers can be added to the surface of tetrapod articles as well.

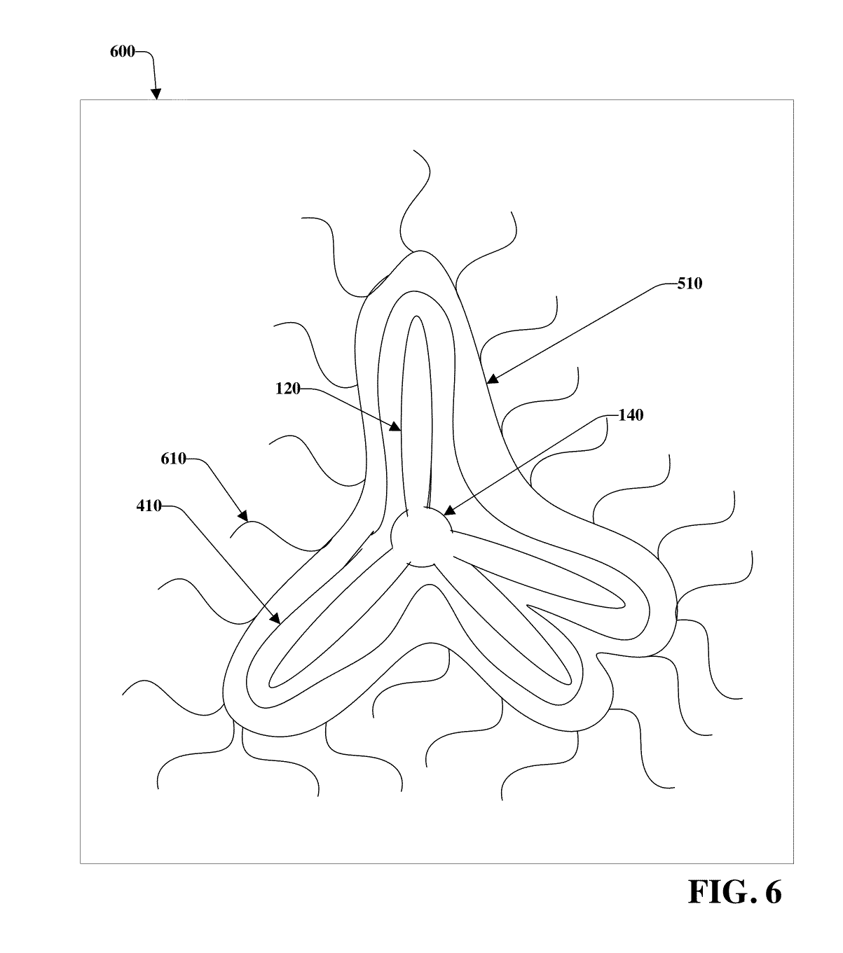

Turning now to FIG. 6, shown is tetrapod article 600 comprising a pairing moiety 610 that can directly surround or overcoat the apex 140 base nanomaterial or the leg 120 base nanomaterial. In another aspect, the pairing moiety 610 can surround or overcoat the nanomaterial layers 410 that overcoats the apex 140 base nanomaterial and the leg 120 base nanomaterial. In yet another aspect, the pairing moiety 610 can surround or overcoat the nanomaterial layers 510 that overcoats the nanomaterial layer 410 that overcoats the apex 140 base nanomaterial and the leg 120 base nanomaterial. A pairing moiety 510 can be functional groups on the tetrapod article 500 surface and other tetrapod article embodiments surface.

In an aspect, functional groups are chemistry groups positioned on the surface of a tetrapod article. In an aspect, tetrapod 600 can coated with any one or more pairing moieties 610 that are chemical groups, or any combination of chemical groups, including, but not limited to, amino groups, carboxyl groups, azide groups, alkyne groups, hydrazine groups, aldehyde groups, aminooxy groups, ketone groups, maleimide groups, thiol groups, or other such chemical groups. In an aspect, a pairing moiety 610 is directly applied to an apex 140 base nanomaterial and leg 120 base nanomaterial. In another aspect, a pairing moiety is directly applied to a nanomaterial layer 410. In another aspect, a pairing moiety is directly applied to a nanomaterial layer 510.

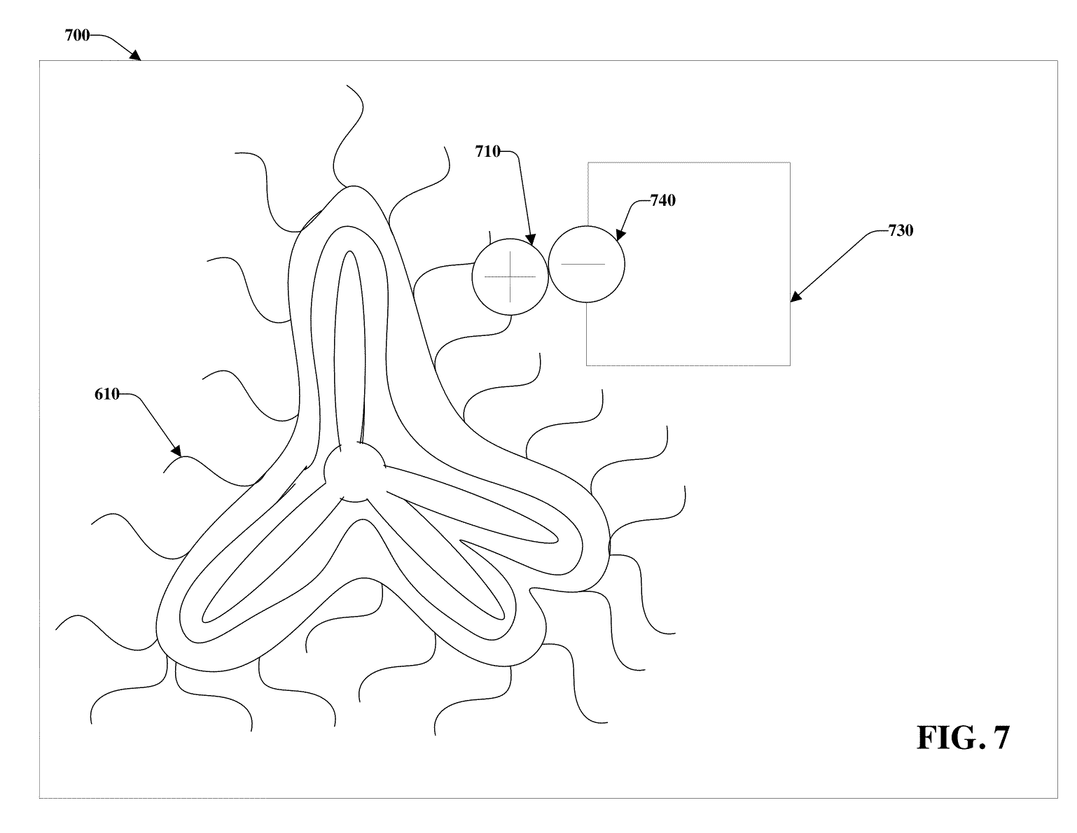

Turning now to FIG. 7, shown is system 700 which demonstrates the charged properties of a tetrapod article comprising a pairing moiety 610 can pair to predefined targeted items 730 through various mechanisms. In an aspect a predefined targeting item 730 can he a biological material such as a cell, antigen receptor, biological marker, peptide, protein, or other such biological materials. The pairing moiety 610 can leave the tetrapod article charged, for instance, the functionalizing of a tetrapod article with an amine group can cause the tetrapod to possess a positive charge 710. Furthermore, a targeted item 730 may possess a negative charge 740, which allows a positive charge 710 located on the surface of a tetrapod article to pair to a negative charge of a targeted item 730. In other aspects, the charge of the tetrapod article can be negative and the charge of the targeted item can be positive. In an aspect, pairing moiety 610 can enable a tetrapod article to pair to a targeting item through other (mechanisms other than charge attractions) pairing mechanisms including, but not limited to, magnetic attraction, attractive forces, bonding, mechanical bonding, electrostatic attraction, chemical bonds, covalent bonds, ionic bonds, hydrogen bonds, Van der Waals' forces, lock and key mechanism, and other such mechanisms.

In an aspect, tetrapod articles can also be paired to genetic material. The tetrapod articles are usually modified to become positively charged by surface modifications of the tetrapod article with NH4+ enabling the tetrapod article to bind to negatively charged negative stranded nucleic acid through electrostatic interactions. This allows for selective gene detection of both RNA, DNA, plasmids, and other such genetic materials. In another aspect, the tetrapod articles can target multiple genetic parameters per single assay by adjusting leg 120 length, leg 120 width, or apex 140 size thereby changing the emission property of the nanomaterials upon excitation with UV or blue light. Furthermore, in an aspect, as opposed to spherical nanocrystals, the tetrapod article's adjustable configuration allows it to pair to more genetic material per singular unit of nanoparticle (e.g. a different genetic material can be bound to each leg 120). In another aspect, the tetrapod articles can be grown as inorganic dendrimers whereby the branched inorganic tetrapod articles are capable of increasing both transfection efficiency.

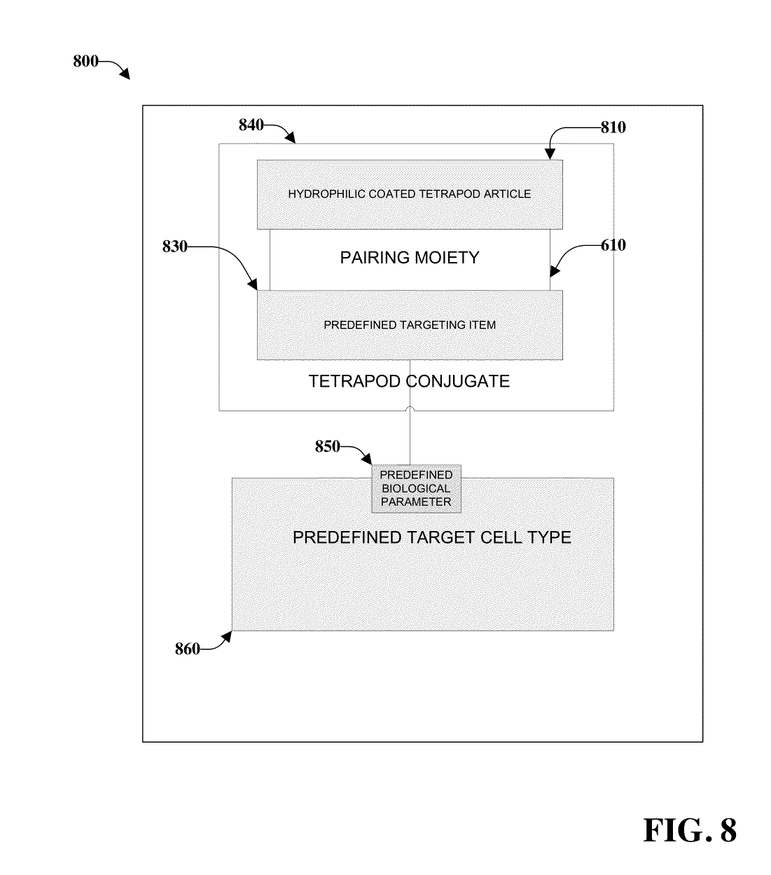

Turning now to FIG. 8, shown is an assay kit 800 comprising; a set of hydrophilic coated tetrapod articles coupled to K pairing moieties that are paired to L predefined targeting items to form a set of tetrapod conjugates that that correspond to M predefined biological parameters and luminesce at respective wavelengths that correspond to the respective predefined target cell types, wherein K, L, and M are integers. In an aspect, hydrophilic coated tetrapod article 810 is a tetrapod article coated with a hydrophilic surface nanomaterial such as amphiphilic ligands or hydrophilic thiol compounds.

In an aspect, hydrophilic coated tetrapod article 810 can contain a hydrophilic coating comprising of Pluronic F127. In these F127 micelles, poly(propylene oxide (PPO) and ply(ethylene oxide)(PEO) serves as the hydrophilic section and poly(ethylene oxide(PEO) acts as the hydrophilic section. In another aspect, the hydrophilic coating can be glass or in part glass, such as silica, SiO, SiO2. Also, the hydrophilic coating can be in total or in part, polymeric oxide, oxide of silicon, oxide of boron, oxide of phosphorus, or a mixture of any one or more oxide. In yet another aspect, the hydrophilic coating can be in part or in whole, any one or mixture of metal silicate, metal borate, or metal phosphate. Other hydrophilic coatings include, but are not limited to, trioctylphosphine (TOPO), ethylene glycol, alkylithio acid, mercaptoacetic acid, or any combination of these coatings.

In another aspect, pairing moiety 610 pairs the hydrophilic coated tetrapod article to a predefined targeting item 830 to form tetrapod conjugate 840. In an aspect, a predefined targeting item 830 is any material possessing a biological function. The predefined targeting item 830 can be any one or more endogenously-synthesized compounds that may influence biological phenomena or represent quantifiable biomarkers. In an aspect, predefined targeting item 830 can include, but is not limited to, any of a variety of biological substances such as antigens, biological markers, blood coagulation factor inhibitors, blood coagulation factors, chemotactic factors, inflammation mediators, intercellular signaling peptides, intracellular proteins, pheromones, pigments, biological toxins and other such biological substances. In another aspect, a predefined targeting item 830 can also be any one or more complex pharmaceutical substances, preparations, or agents of organic origin, usually obtained by biological methods or assay.

Furthermore, predefined targeting item 830 can be any one or more of an antibody, monoclonal antibody, nucleic acid, protein, polysaccharide, sugar, peptide, drug, oligomeric nucleic acid, or monomeric nucleic acid. The hydrophilic coated tetrapod article paired to a predefined targeting item 830 is known as a tetrapod conjugate 840. In an embodiment, a tetrapod conjugate 840 is used in a biological or medical analysis system. In an aspect, a tetrapod conjugate 840 is used to evaluate and diagnose disease states of tissue, either in vivo or in vitro. In another aspect, the tetrapod conjugate 840 is used for medical imaging applications. In yet another aspect, the nanoplex is used for diagnostic assay kits. In an instance, the tetrapod conjugate 840 contacts one or more predefined biological parameters 850. Upon contact, the tetrapod conjugate 840 is exposed to a wavelength of light (e.g. ultraviolet light of a shorter wavelength than the respective emission wavelength of the nanoplex) that causes the nanoplex to luminesce and can be detected based on changes in fluorescence.

In an embodiment, the tetrapod conjugate 840 can identify a predefined biological parameter 850. In an aspect, tetrapod conjugate 840 can identify a predefined biological parameter 850 by tagging the predefined biological parameter 850. Tagging occurs when one or more tetrapod conjugate 840 is matched with one or more respective predefined biological parameter 850. A tetrapod conjugate 840 can pair with a predefined biological parameter 850 by various mechanisms including, but not limited to, magnetic attraction, attractive forces, bonding, mechanical bonding, electrostatic attraction, chemical bonds, covalent bonds, ionic bonds, hydrogen bonds, Van der Waals' forces, lock and key mechanism, and other such mechanisms.

The tagging of a biological parameter by a tetrapod conjugate 840 can occur in many ways, one such way occurs through an antibody-receptor bonding. An antibody is a blood protein produced in response to and counteracting a specific antigen. Antigens are foreign molecular structures (e.g. a surface protein on a virus, a surface protein on a bacteria, an animal toxin, a surface protein on human tissue cells, etc.) and accordingly particular antibodies have an affinity for particular antigens and will bind to such antigen upon contact. An antibody can also associate with a receptor (located extracellularly or intracellularly) often times through an antibody-receptor bond. An antibody can perform various functions, all simply by binding to an antigen and/or a receptor. For instance, many viruses and some bacterial toxins affect cells by binding to receptors on cells. This either alters cell function (in the case of toxins) or allows entry into the cell (in the case of viruses). An antibody that binds to an antigen can block the virus or bacterial toxin and stop such virus or bacterial toxin from binding to a cell. For purposes of a diagnostic assay kit, an antibody that occupies a receptor site will luminesce and act as a tagging marker for such receptor site.

In an aspect, where the assay kit is a flow-based assay, the antibody serves as biological molecule that binds to the desired biological parameter targeted for detection in the sample. Flow based detection happens whereby the number of events correlates with the number of cells paired to the tetrapod conjugate 840 through the antibody. The tetrapod conjugate 840 would be composed of an 1:1 ratio of antibody to tetrapod article, therefore allowing for accurate measurement of concentration based on the number of events detected on a flow reading. In addition, if a predefined target cell type 860 is useful for phenotyping, which requires more than one biological parameter for detection; a cocktail of tetrapod conjugate 840 can be used whereby each wavelength of tetrapod article is paired to a specific antibody marking a specific biological parameter on a predefined target cell 860. The combination of tetrapod articles paired to predefined targeting item 830 will allow an flow event to include all parameters at the same time with minimal compensation due to the quantum dots having narrow emission wavelengths.

In another embodiment, the predefined target cell type 860 is any type of cell associated with one or more predefined targeting item 830. Cells are the fundamental structural, and functional units or submits of living organisms. Researchers (e.g. cell biology researchers, cancer researchers, etc.) are interested in studying cells for physiological properties, structure, cellular organelles, function, interaction with environment, life cycle, division, death, differentiating between cell types, and other such interests. In an aspect, assay kit 800 provides an efficient and effective assay to identify biological parameters associated with predefined target cell type 860. In an aspect, predefined target cell type 860 can include, but is not limited to, transformed cell lines, hybrid cell lines, tumor cell lines, stem cells, and other such cells. In another aspect, target cell type 150 can include any one or more of the cell types in the human body and animal bodies (e.g pig, rat, sheep, monkey, mouse, rat, etc.).

Further, predefined target cell types 860 can be any one or more gland cells such as exocrine secretory epithelial cells (e.g. salivary gland serous cell, sebaceous gland cell, etc.), hormone secreting cells (e.g. anterior pituitary cells, intermediate pituitary cell, magnocellular neurosecretory cells, gut and respiratory tract cells, thyroid gland cells, parathyroid gland cells, parathyroid gland cells, adrenal gland cells, leydig cell of testes, theca interna cell of ovarian follicle, cirous luteum cell, etc.), epithelial cells lining closed internal body cavities, ciliated cells with propulsive function, integumentary system cells (e.g. keratinizing epithelial cells, wet stratified barrier epithelial cells), nervous system cells (e.g. sensory transducer cells, autonomic neuron cells, sense organ and peripheral neuron supporting cells, central nervous system neurons and glial cells, lens cells, etc.), cells derived primarily from mesoderm (e.g. metabolism and storage cells, barrier function cells (lung, gut, exocrine glands, urogenital tract), kidney cells, extracellular matrix cells, contractile cells, blood and immune system cells, pigment cells, germ cells, nurse cells, interstitial cells), and other such cell types.

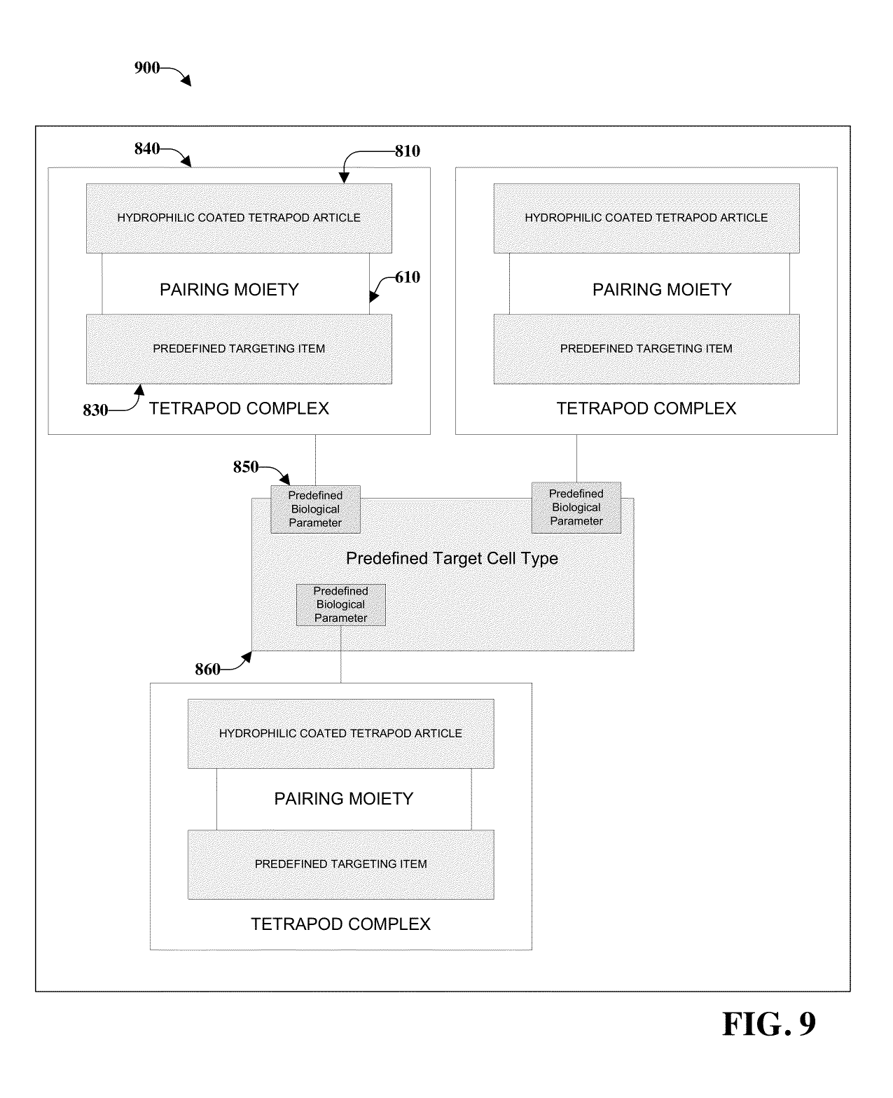

Turning now to FIG. 9, shown is an assay kit 900 comprising; a set of hydrophilic coated tetrapod articles coupled to K pairing moieties that are paired to L predefined targeting items to form a set of tetrapod conjugates that that correspond to M predefined biological parameters and luminesce at respective wavelengths that correspond to the respective predefined target cell types, wherein K, L, and M are integers. In an aspect, assay kit 900 illustrates multiplexing. Multiplexing in this instance, is the simultaneous amplification, labeling and detection of many different targets with one single excitation source.

In an aspect, unique tetrapod articles allow for the tracking of more than one biological parameters given the distinguishing characteristics afforded by these articles. Furthermore, the tetrapod article is capable of the arm length and width being adjustable as shorter, longer, thicker or thinner, which allows for the identification of various parameters through the presentation of unique spectral signatures based on one excitation source such as electromagnetic radiation, UV light, x-ray, visible light, infrared light, blue light, or other such excitation sources within these wavelength light ranges. The enhanced optical properties are also more robust when compared to standard spherical luminescent quantum dots with a smaller width at half maximum wavelength.

Furthermore, multiple analytes can be identified concurrently by use of many tetrapod articles, each tetrapod article respectively, may emit at various wavelengths due to varying arm lengths, arm widths, apex diameters, material compositions, and productions of several tetrapod articles of such varying arm lengths, arm widths, apex diameters, all of which are excitable by one single excitation source. This offers a significant advantage in that each tetrapod quantum dot is capable of possessing a unique spectral signature by adjusting any of four features, arm length, arm width, number of arms, or apex diameter. Conversely, a spherical quantum dot only possesses a unique spectral signature based on the adjusting of one feature; adjustment of the spherical diameter. This difference allows for greater multiplexing applications available for tetrapod articles due to the availability of more tetrapod articles with unique spectral signatures versus spherical quantum dots.

In one embodiment, the assay 900 uses the idea of attaching many colors of tetrapod articles to different respective antibodies in order to obtain a distinctive combination of wavelengths. If five different antibodies for instance have five different tetrapod articles paired to its surface with five different associated wavelengths, each tetrapod article emitting at a different wavelength, then an instrument can detect the nature of such target, by reading the live wavelengths emitted from such target in sequence. Likewise, sequence variations of tetrapod articles with different wavelengths attached to targets can result in hundreds of parameters being tagged in a single instance.

In another embodiment, each tetrapod can be produced in such a customized manner to possess different arm lengths, different arm widths, different core and shell materials, each arm and/or apex of which possess the ability to emit at different wavelengths all the while belonging to the same tetrapod articles. Thus, in an aspect, hundreds of tetrapod articles may be customized to track hundreds of targets in one single sample.

One of the selective optical properties of tetrapod articles that serve an important biological advantage is that the half maximum width of tetrapod articles are smaller than the conventional spherical quantum dots: the benefits of this is that it's easier to decipher more parameters when conducting real time analysis or multiplexing work due to more emission spectrums that can fit for a defined range of detection wavelengths. In an aspect there are many applications that can be created by pairing tetrapod articles to biological materials. For instance, by binding tetrapod articles to both monoclonal and polyclonal antibodies, novel assay kits can be created that will help create enhanced biological detection tools that work on the premise of tetrapod articles replacing existing biological dyes bound to antibodies. By using non-spherical tetrapod articles we achieve increased sensitivity, require less sample volumes, and measure multiple antigens in real time. Existing diagnostic techniques in the biological sciences are enhanced by these properties including but not limited to: flow cytometry applications, IHC, lateral flow assays, Western Blot, ELISA, microarray, PCR arrays, peizoarrays, and other such diagnostic tests.

In an aspect, assay 900, can be created by pairing_tetrapod articles to monoclonal or polyclonal antibodies. By using tetrapod article increased sensitivity, less sample volumes, and simultaneous multiple antigen measurements in real time can be achieved. Existing diagnostic techniques in the biological sciences are enhanced by these properties including but not limited to: flow cytometry applications, IHC, Western Blot, ELISA, microarray, PCR arrays, peizoarrays, and other such techniques.

Additionally, the assay 900 also has the ability to cause the identification of hundreds of analytes in a sample. Such application may go forth to encompass assays in which multiple analytes can be identified concurrently by use of many tetrapod articles, each tetrapod article respectively, may emit at various wavelengths due to adjustments to leg 120 lengths, leg 120 widths, apex sizes, or material compositions, whereby all the tetrapod articles are excitable by one single excitation source and allowing for multiplexing. Multiplexing is the simultaneous amplification, labeling and detection of many different targets with one single excitation source. The tetrapod articles herein allow for the tracking of several parameters given the distinguishing characteristics afforded by these particles. In one embodiment, the assay 900 utilizes the feature of attaching many colors (unique tetrapod articles with varying spectral signatures) to different respective antibodies in order to obtain a distinctive set of spectral signatures.

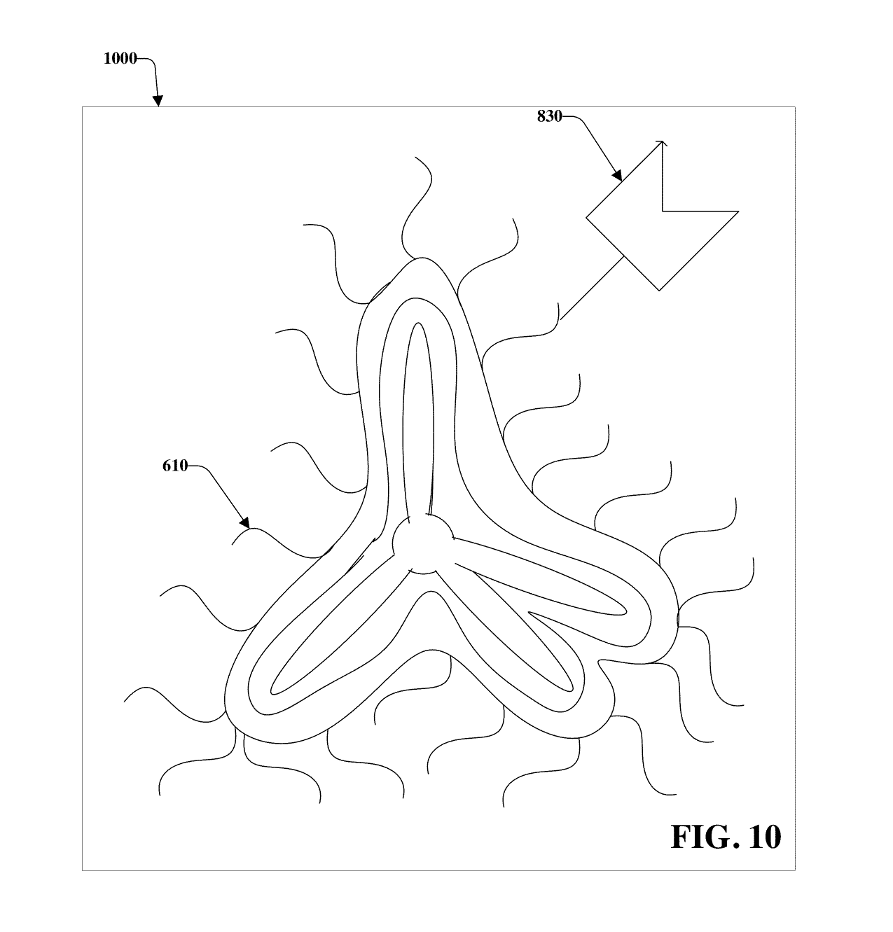

Turning now to FIG. 10, shown is tetrapod article 1000, which illustrates a tetrapod article, paired to a predefined targeting item 830. In an aspect, the pairing of a tetrapod article to a predefined targeting item 830 can lead to increased cellular uptake of the tetrapod article paired to the predefined targeting item 830 at lower toxicity profiles. In an aspect, the tetrapod article 1000 can comprise non-toxic base nanomaterials and layer nanomaterials such as phosphorus and non-cadmium based materials for safe use within a subject (e.g. human or animals). In an aspect, the predefined targeting item 830 can be an antibody or drug. The tetrapod article can be paired to a drug through a customized pairing moiety 610 that accounts for unique drug features such as drug structure, pharmacokinetics, toxicity, route of administration, and other such drug properties.

Additionally, tetrapod articles, unlike spherical nanocrystals, can present greater cellular uptake due to its unique shape. In an some instances, cells in the living system can make use of polyanionic receptors as a method of conducting either receptor mediated endocytosis or phagocytosis to uptake biological materials such as antibodies. Thus, a tetrapod article paired to an antibody can be uptaken by cells through such endocytosis and phagocytosis. Furthermore, by affecting the overall spatial charge distribution around the tetrapod article (due to its unique configuration of apex and legs) the cell can better uptake the tetrapod article via electrostatic attractive forces. Also, the tetrapod article can be paired to one or more tetrapod articles to form an inorganic tetrapod dendrimer with branches created by the legs 120. An inorganic tetrapod dendrimer can allow for greater transfection of a drug other such targeting item 830 leading to a more favorable pharmacokinetic profile and therapeutic index to effectively maximize therapeutic efficacy of the drug in a subject while minimizing systemic and regional side effects in the subject.

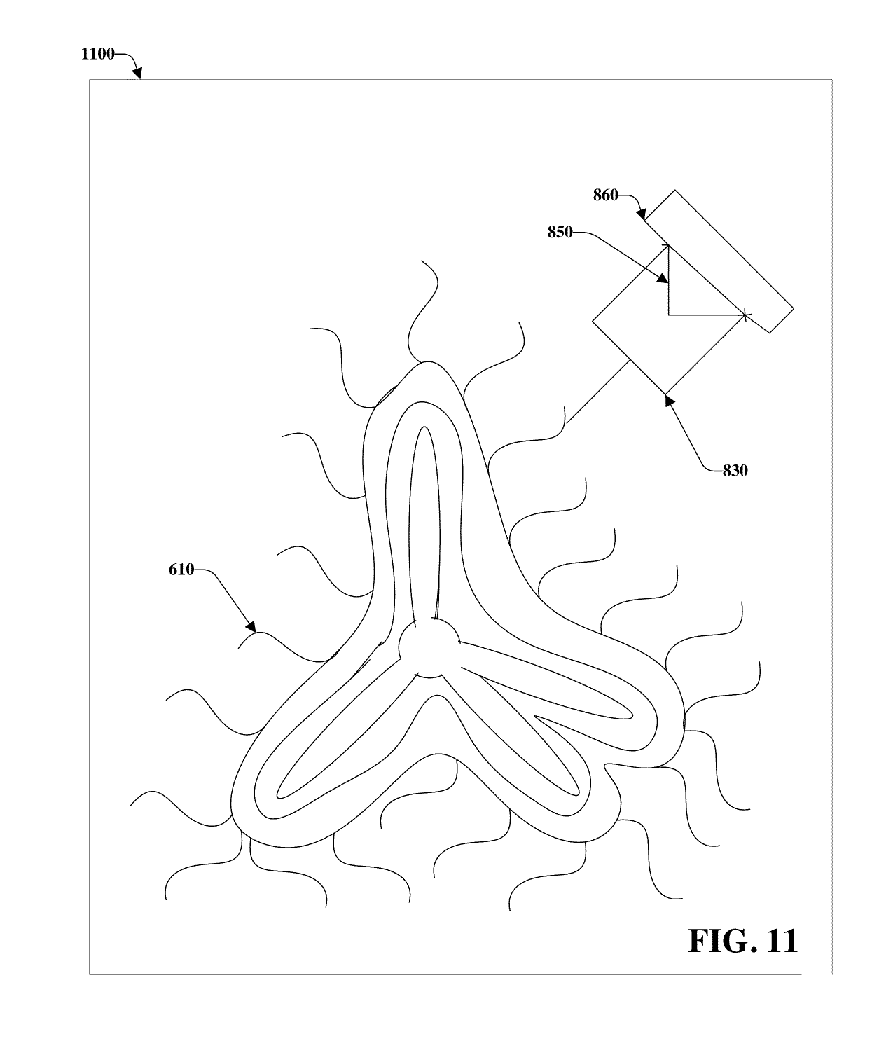

Turning now to FIG. 11, shown is tetrapod article 1100, which illustrates a tetrapod article, paired to a predefined targeting item 830. In an aspect, the predefined targeting item 830 can pair to a predefined biological parameter 850 associated with a predefined target cell type 860. For instance, in an aspect, predefined targeting item 830 is a protein, predefined biological parameter 850 is a cellular receptor associated with the protein, and predefined cell type 860 is a cell whereby the receptor presents itself (e.g. intracellular receptor, extracellular receptor, etc.). Thus the tetrapod article can serve as a luminescent detection agent, the protein is a targeting agent that corresponds to a specific cellular receptor. Once the protein binds to the receptor the tetrapod article can be excited and produce a spectral signature. The more biological parameters tagged with respective tetrapod articles, the greater the likelihood that a specific cell type is identified. In another aspect, the targeting via a tetrapod article can he used to track the movement of drugs, antibodies, proteins and other biological materials movement in a subject.

The ability to modify the surface chemistry of the tetrapod article allows for selective targeting of specific cell types including but not limited to cancer cells, neurons, and other cells within a subject. In an instance, tissue factor receptors on the surface of pancreatic cells can enhance uptake of tetrapod articles by the pancreatic cancer cells. Furthermore, in an aspect, the tetrapod articles can utilize magnetics to enhance its targeting abilities. In an instance the apex 140 can be comprised of a luminescent semiconductor nanomaterial and the leg 120 can be comprised of magnetic nanomaterials such as iron oxide in order to allow for stronger attractive forces to guide the targeting feature of the luminescent tetrapod articles.

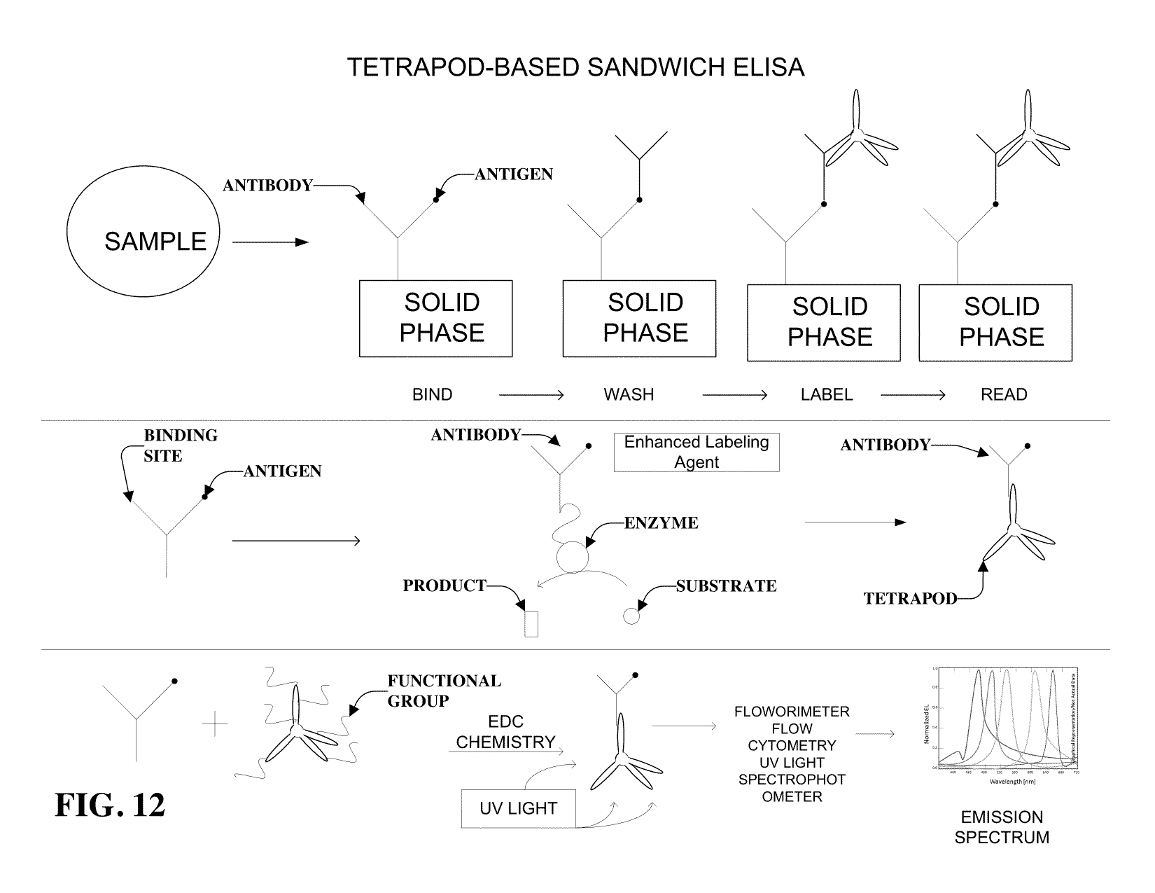

Turning now to FIG. 12, shown is a tetrapod article based sandwich ELISA. FIG. 12 illustrates one example of a diagnostic application using inorganic tetrapod articles for biological applications. A sandwich ELISA utilizes an unknown amount of antigen immobilized on a solid support by a primary antibody. A secondary (also known as detection antibody is then bound to the primary antibody antigen paired to a tetrapod article. The secondary antibody is typically linked to an enzyme through bioconjugation mechanisms whereby the secondary antibody conjugated to an enzyme cleaves an enzymatic substrate to produce a visible signal, which indicates the quantity of antigen present in the sample based on a standard curve. The tetrapod article can be paired to the secondary antibody and subsequently excited by UV light to produce a visible signal. This aspect whereby an antibody (e.g. polyclonal or monoclonal) is bound to a tetrapod article allows for the creation of enhanced applications that include but are not limited to ELISA, sandwich ELISA, PCR, Western Blot, flow cytometry, FRET analysis, microarray, peizoarrays, Southern Blot, lateral flow assays, single antibody immunoflouresce measurements, and drug and gene delivery.

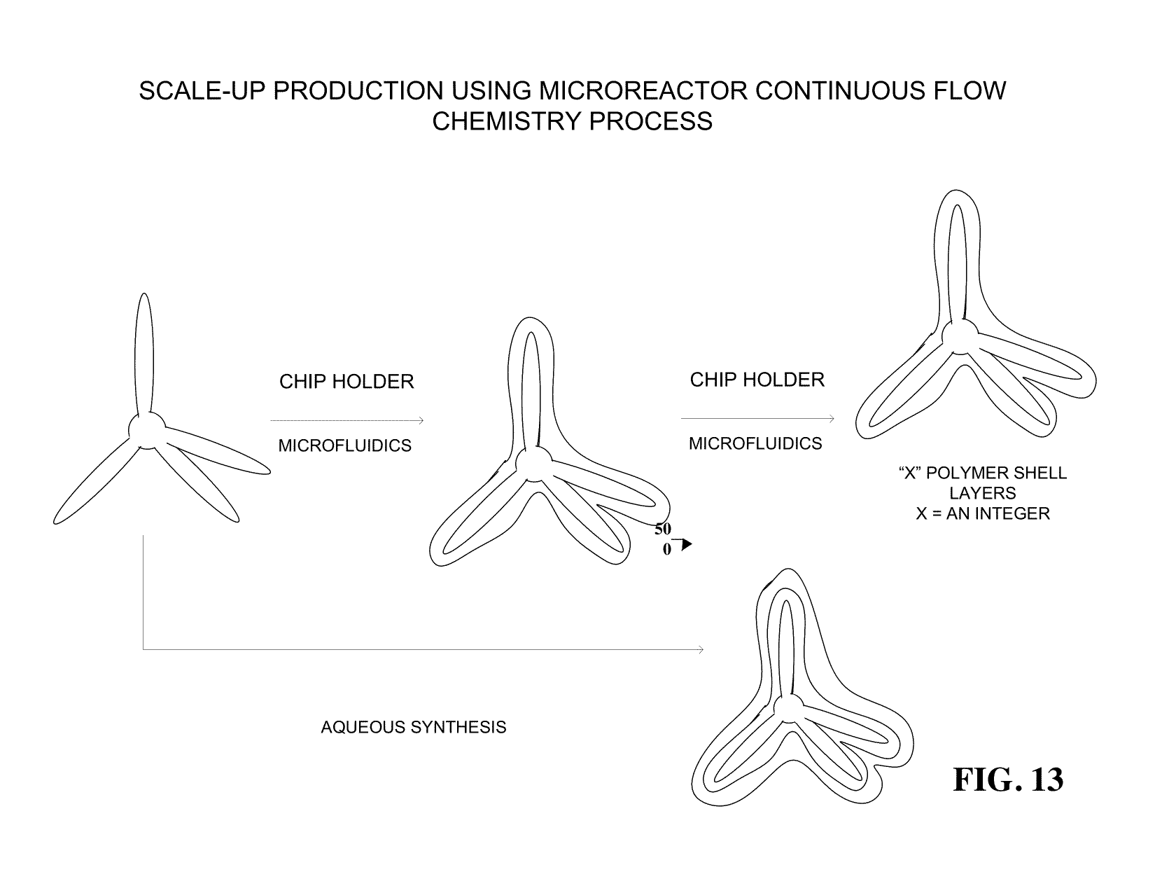

Turning now to FIG. 13, shown is a tetrapod article produced using a microreactor continuous flow chemistry process. In an aspect the microreactor process can produce an apex 140 base nanomaterial, an leg 120 base nanomaterial, a nanomaterial layer 410, and all disclosed tetrapod articles herein in large-scale batch quantities. Additionally numerous nanomaterial layer 410 overcoats can be added to the tetrapod articles. In another aspect, a functionalized tetrapod article comprising pairing moieties can be produced using the microreactor process. All such steps can occur in a flow chemistry microreactor.

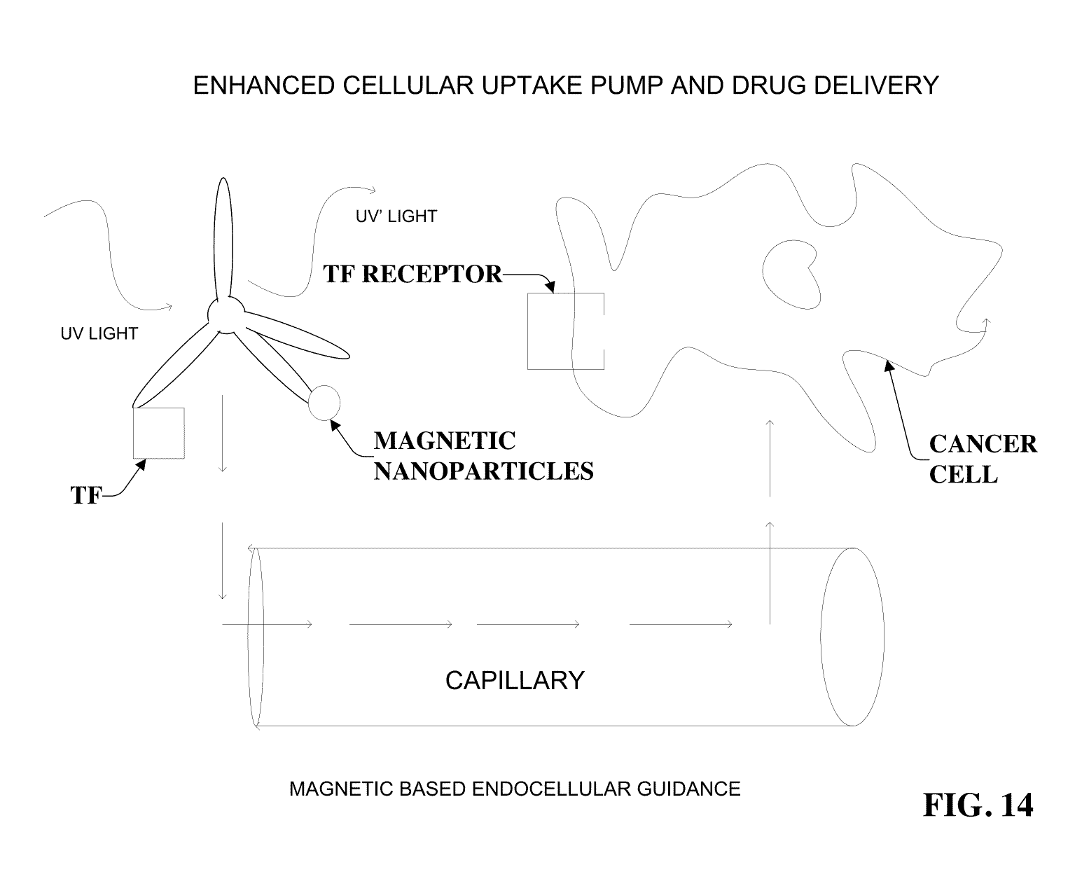

Turning now to FIG. 14, shown is an enhanced cellular uptake pump and drug delivery system. FIG. 14 illustrates tetrapod articles used in targeted drug delivery applications. In an aspect, the tetrapod articles can be used to accomplish multimodal targeting whereby, the tetrapod articles are composed and paired to magnetic material compositions such as iron oxide to bind to tissue factor antibodies allowing for selective targeting of tetrapod article to cancer cells that over-express tissue factor receptor. This external guidance, whereby magnetized tetrapod articles are trafficked to cancer cells that over express tissue factor receptor offers new solutions to treating various cancers. In an aspect, this external guidance can be administered intravenously with drugs, whereby the tetrapod article is paired to the drug as well and localized to the cancer cell regions in a subject.

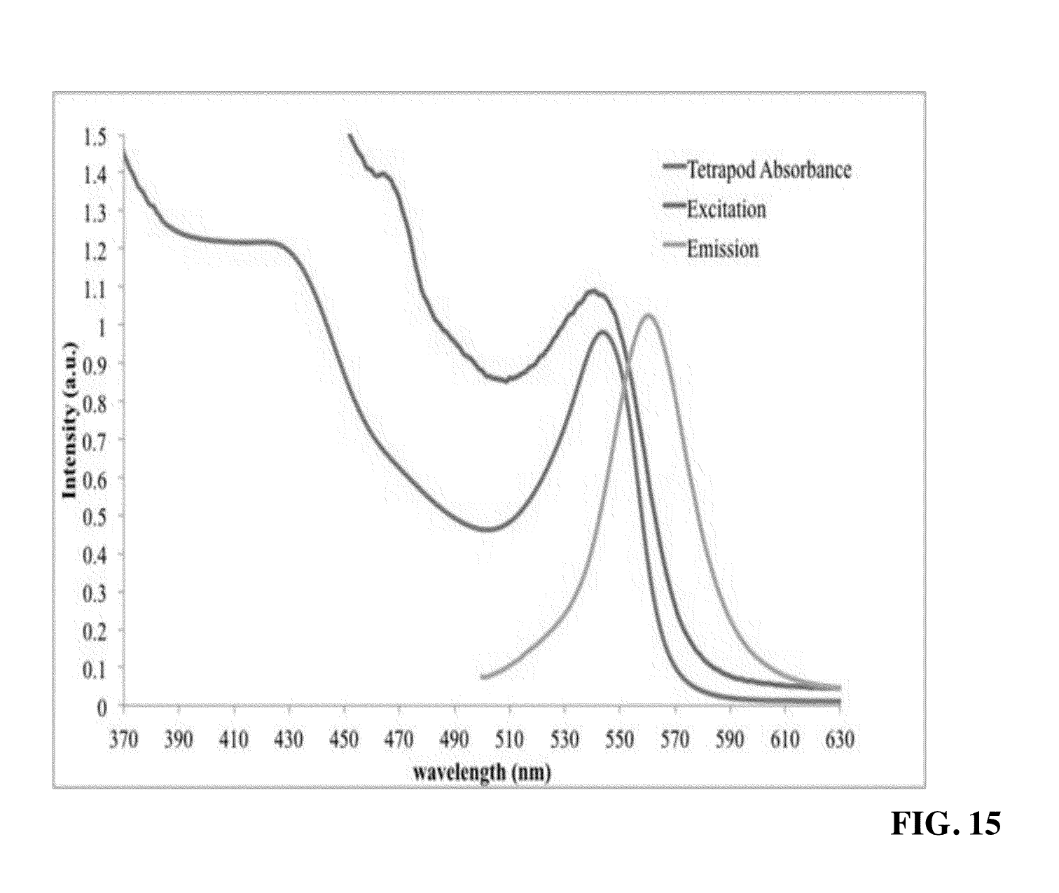

Turning now to FIG. 15, illustrated is the absorbance, excitation and emission results of tetrapod articles. In an aspect, the figure demonstrates the wavelength on the x-axis plotted against the intensity on the y-axis, whereby the intensity is listed in absorbance units. The red curve represents the wavelength of the excitation source, which has a peak of approximately 540 nm and an intensity of approximately 1.1 absorption units. The blue curve represents the absorbance of the tetrapod article, which occurs at a wavelength of approximately 545 nm, as demonstrated by the blue peak, and demonstrates an intensity of approximately 1 absorbance unit. The green curve represents the emission wavelength of the tetrapod articles, which occurs at approximately 570 nm and demonstrates an intensity of between 1 and 1.1 absorption units.

This data demonstrates that an unbound tetrapod article comprising of a cadmium base material apex and a cadmium base material leg that is excited by an excitation source that excites at 540 nm, absorbs the emitted excitation energy at 545 nm. Furthermore, the tetrapod article emits the energy at 560 nm. Thus the unbound tetrapod article emits (green peak) more energy than it absorbs (red peak). Accordingly a tetrapod article that absorbs excitation energy at a wavelength of 560 nm can be excited by an excitation source that emits excitation energy at below 560 nm and can absorb energy (e.g. UV light) at less than 560 nm.

The conjugations occurred by tetrapod conjugation with folic acid was preceded using the carbodi-imide conjugation method. Briefly, 4 mg of water-soluble tetrapods were dispersed in 2 ml PBS (1 mM, pH 7.4). Freshly prepared EDC (1 mM, 100 microliters) and NHS (1 mM, 100 microliters) where added to the water-soluble tetrapod and then allowed to react in dark for an hour. The excess EDC and NHS were washed by centrifugation under 1500 rpm for 10 minutes. Folic Acid in PBS (12.8 mM, 100 microliters) was added to the tetrapod solution and then was allowed to react for 4 hours with the activated tetrapods. After the incubation period, the tetrapods were centrifuged and washed to remove the excess and unconjugated tetrapods. The pellet was re-dispersed into 2 ml PBS and stored at 4 degree Celsius for further characterization.

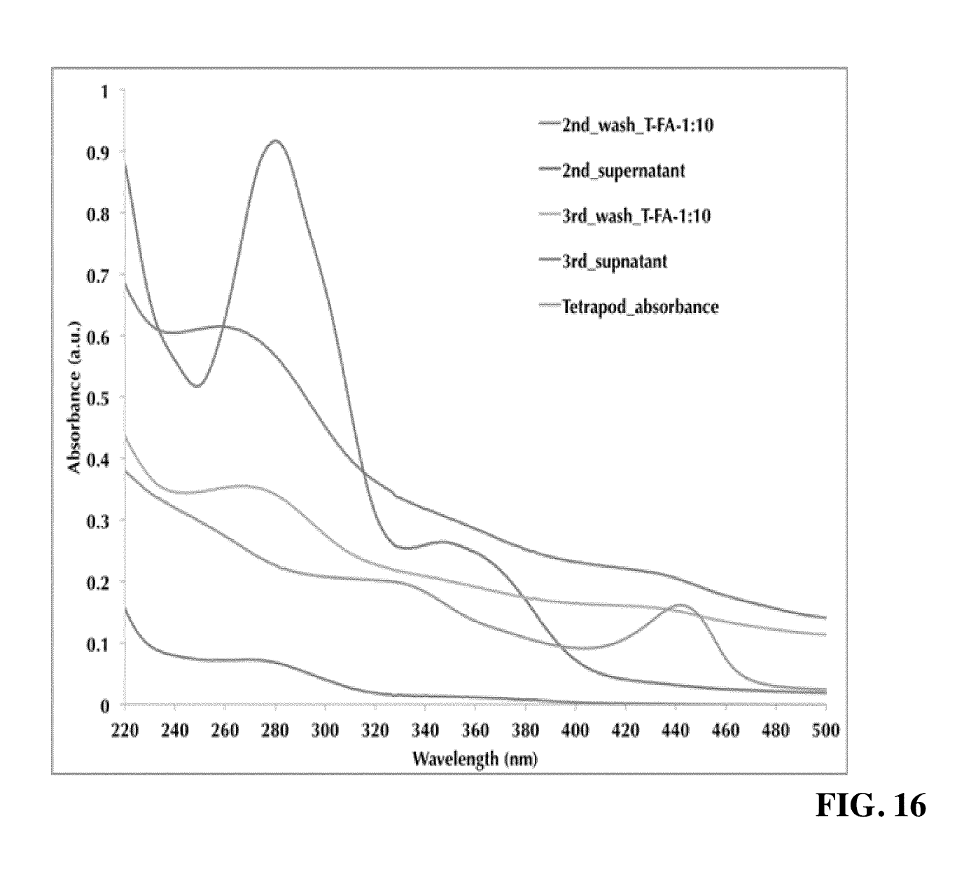

Turning now to FIG. 16, illustrated is the absorbance data (in absorbance units) of tetrapod articles, folic acid, and tetrapod articles conjugated to folic acid (referred to as Tetrapod-FA). Subsequent to the tetrapod and folic acid conjugation, the solution was centrifuged and the absorbance of the supernatant (unpaired folic acid) was measured for absorbance. Thus the dark blue curve represents the Tetrapod-FA conjugate (conjugated at a 1:10 ratio of Tetrapod to FA) after the first centrifuge (also known as the second wash). The blue curve shows two peaks whereby the first blue peak occurs between 420-470 nm and the second blue peak occurs between 420-470 nm for the Tetrapod-FA conjugate. The first dark blue peak between 420-470 nm represents the tetrapod article of the Tetrapod-FA conjugate and the second blue peak between 260-270 nm represents the FA of the Tetrapod-FA conjugate. We demonstrate that through repeated centrifugation and wash steps, we can reduce the unbound folic acid in our sample leaving only bound Tetrapod-FA conjugate present. This is indicated by the absence of the purple curve having the folic acid absorption peak present in the second supernatant that followed the second wash step as indicated by the red curve. This method serves as useful way to isolate only bound Tetrapod-FA material suitable for biological applications.

Additionally, the supernatant was analyzed for absorbance data, whereby the wavelength of the supernatant after the second wash was approximately 270 nm thereby possibly indicating that unbound folic acid remained in the supernatant (also meaning there was an absence of tetrapod articles left in the supernatant). Accordingly, after the second centrifuge (also known as third wash) of the material, the supernatant showed a very small peak (indicated by the purple curve) at between 270-285 nm indicating almost no folic acid was unbound after this 3^{rd} wash step. The light blue curve indicates unbound tetrapod articles which demonstrate a peak at between 430 and 450 nm. The light green curve is the absorbance measure of the Tetrapod-FA sample after the 3^{rd} wash step which revealed a slight blue shift of the peaks of approximately 10 nm.

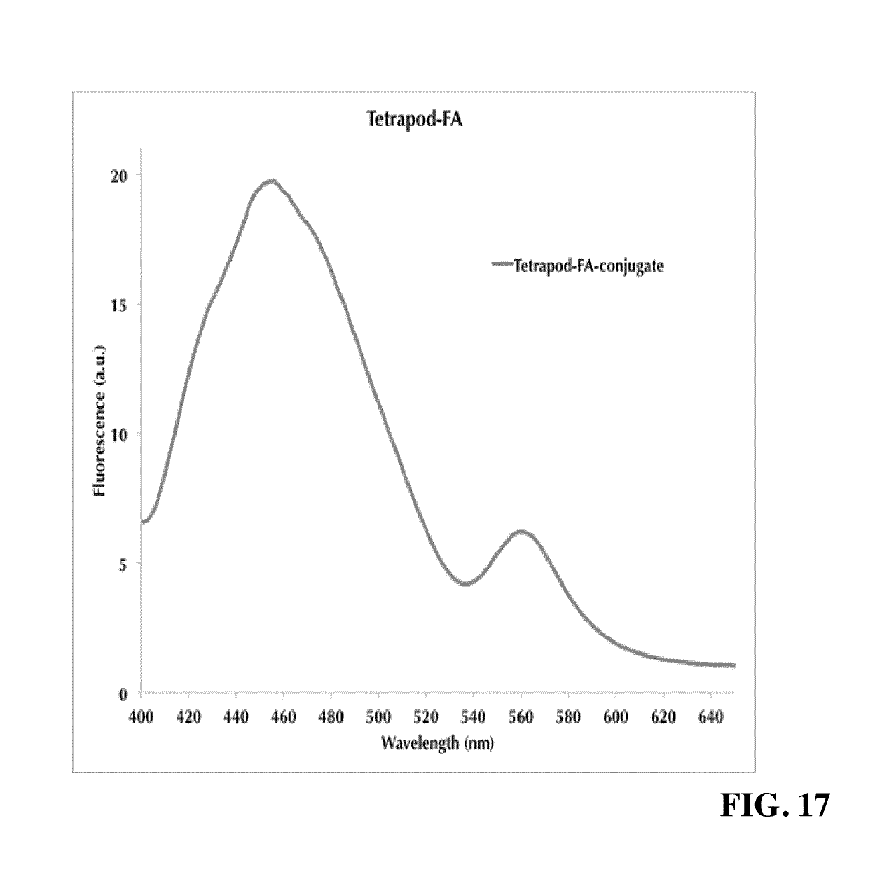

Turning now to FIG. 17, illustrated are the results after the 3^{rd} wash step whereby the fluorescence (Y-axis) of the tetrapod-FA conjugate is demonstrated. The graph does the fluorescence of the folic acid (peak at approximately 455 nm) and a tetrapod article (peak at approximately 560 nm) for the Tetrapod-FA conjugated material. This demonstrates a blue shift of the tetrapod fluorescence due to conjugations.

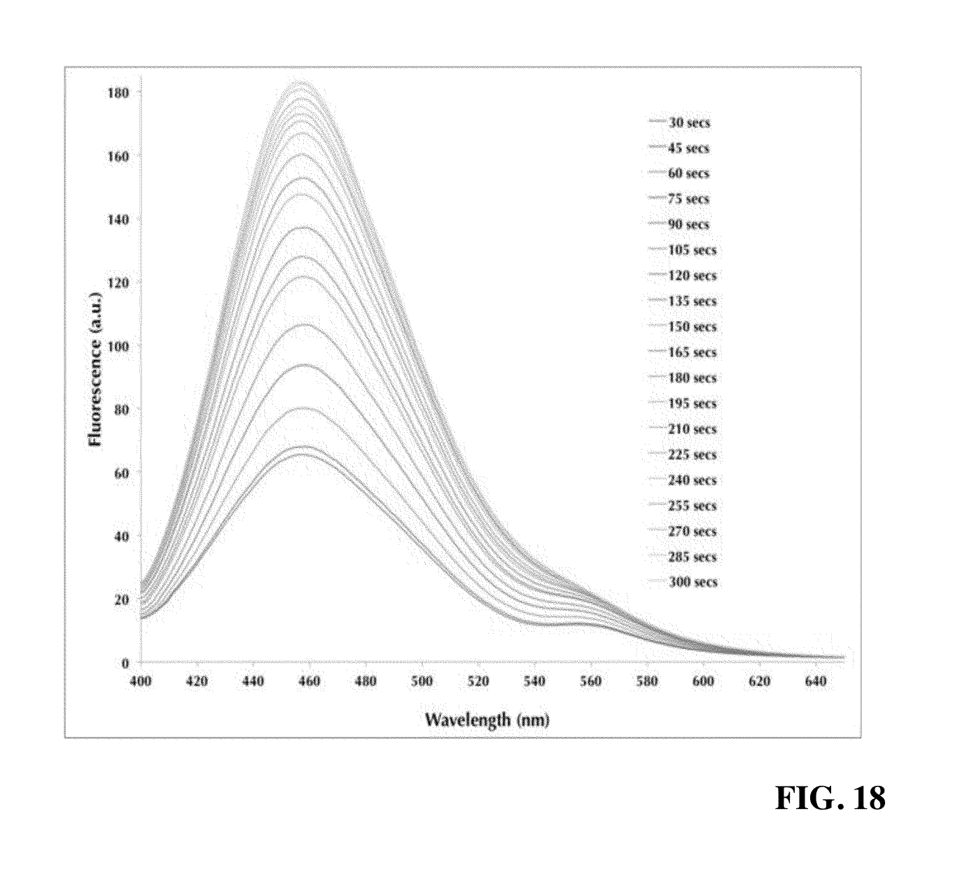

Turning now to FIG. 18, illustrated is a plot of the fluorescence of the Tetrapod-FA conjugate with respect to time. The photo-oxidation of the folic acid causes the increase in fluorescence of the folic acid with time. This can be easily interpreted from the graph above. However, we can see a noticeable though small change in the fluorescence peak of the tetrapod articles at 560 nm. This might be attributed to the FRET taking place between the folic acid and tetrapods, since one peak of absorbance of tetrapod (465 nm) lie in vicinity to the fluorescence of folic acid. The change in fluorescence of folic acid is high enough to mask the effect of FRET though.

In summary, disclosed are tetrapod articles, assay kits, and terapod article applications. The embodiments are non-limiting and nothing in this specification is intended to limit the scope of the present invention. The described embodiments may be modified or varied, without departing from the invention. All examples presented are representative an are non-limiting.

What is claimed is:

1. A tetrapod article, comprising:
an apex base nanomaterial; and
X leg base nanomaterial protruding from the apex, wherein X is an integer.

2. The tetrapod article of claim 1, comprising Y nanomaterial layers overcoating the apex base material and the leg base nanomaterial protruding from the apex, wherein Y is an integer.

3. The tetrapod article of claim 1, comprising an apex with four leg base nanomaterials protruding from the apex.

4. The tetrapod article of claim 2, wherein the apex, or the leg is coated with a hydrophilic coating.

5. The article of claim 2, wherein the nanomaterial layers overcoating is a hydrophilic coating.

6. The tetrapod article of claim 1, comprising a pairing moiety surrounding the apex or the leg.

7. The tetrapod article of claim 2, comprising a pairing moiety surrounding the nanomaterial layers.

8. The tetrapod article of claim 6, wherein the pairing moiety is paired to a predefined targeting item, and the targeting item corresponds to one or more predefined biological parameters.

9. The tetrapod article of claim 7, wherein the pairing moiety is paired to a predefined targeting item, and the targeting item corresponds to one or more predefined biological parameters.

10. The tetrapod article of claim 1, wherein the apex base nanomaterial and leg base nanomaterial is a II-VI semiconductor or a III-V semiconductor.

11. The tetrapod article of claim 2, wherein the apex base nanomaterial, leg base nanomaterial, or the nanomaterial layers is a II-VI semiconductor or a III-V semiconductor.

12. The tetrapod article of claim 2, wherein the apex base nanomaterial, the leg base nanomaterial, or the nanomaterial layers is at least one of CdS, CdSe, MgSe, MgTe, MgS , ZnS, ZnTe, ZnSe, CaS, CaSe, SrS, SrSe, SrTe, HgS, HgSe, HgTe, BaS, BaSe, BaTe or CdTe.

13. A assay kit comprising:
a set of hydrophilic coated tetrapod articles coupled to K pairing moieties that are paired to L predefined targeting items to form a set of tetrapod conjugates that that correspond to M predefined biological parameters and luminesce at respective wavelengths that correspond to the respective predefined target cell types, wherein K, L, and M are integers.

14. A branched tetrapod article comprising, a tetrapod article paired to one or more tetrapod articles.

15. The tetrapod article of claim 1, further comprising an apex base nanomaterial paired to both end points of each respective leg base nanomaterial.

16. The tetrapod article of claim 6, wherein the pairing moiety is any one of a thiol moiety, F127COOH, alkyl group, propyl group, N-(3-aminopropyl)-3-mercapto-benzamide, 3-aminopropyl-trimethoxysilane, 3-mercaptopropyltrimethoxysilane, 3-maleimidopropyl-trimethoxysilane, or 3-hydrazidopropyl-trimethoxysilane, diacetylenes, acrylates, acrylamides, vinyl, and styryl.

17. The tetrapod article of claim 7, wherein the pairing moiety is any one of a thiol moiety, F127COOH, alkyl group, propyl group, N-(3-aminopropyl)-3-mercapto-benzamide, 3-aminopropyl-trimethoxysilane, 3-mercaptopropyltrimethoxysilane, 3-maleimidopropyl-trimethoxysilane, or 3-hydrazidopropyl-trimethoxysilane, diacetylenes, acrylates, acrylamides, vinyl, and styryl.

18. The tetrapod article of claim 8, wherein the targeting item is any one of an antibody, a nucleic acid, a protein, a polysaccharide, a small molecule, avidin, streptavidin, a biotin, a antidigoxiginen, a monoclonal antibody, a polyclonal antibody, a nucleic acid, monomeric nucleic acid, 40 oligomeric nucleic acid, a protein, a polysaccharide, a sugar, a peptide, a drag, carbohydrate, ligands.

19. The tetrapod article of claim 9, wherein the targeting item is any one of an antibody, a nucleic acid, a protein, a polysaccharide, a small molecule, avidin, streptavidin, a biotin, a antidigoxiginen, a monoclonal antibody, a polyclonal antibody, a nucleic acid, monomeric nucleic acid, 40 oligomeric nucleic acid, a protein, a polysaccharide, a sugar, a peptide, a drug, carbohydrate, ligands.
